(19) United States
(12) Patent Application Publication
(10) Pub. No.: US 2007/0015988 A1
(43) Pub. Date: Jan. 18, 2007

(54) Docking device

(76) Inventors: Robert M. Woskow, Encino, CA (US); Harold B. Rose, Tarzana, CA (US)

Correspondence Address: COOLEY GODWARD KRONISH LLP;ATTN: PATENT GROUP, THE BOWEN BUILDING, 875 15TH STREET, N.W. SUITE 800, WASHINGTON, DC, 20005-2221, US

(21) Appl. No.: 11/152,542

(22) Filed: Jun. 14, 2005

(51) Int. Cl. A61B 5/05

(52) U.S. Cl. 600/407

(57) ABSTRACT

A facility for coupling different types of catheters to an imaging system is provided. The facility comprises a docking module and a plurality of couplers for coupling the different types of catheters to the imaging system. The docking module provides a mechanical and electrical interface between a catheter and the imaging system. The plurality of couplers enables the different types of catheters having different mechanical and electrical coupling structures, to engage and interact with the docking module.

### BACKGROUND OF THE INVENTION

The present invention relates generally to medical imaging systems and more particularly, to docking devices for use with imaging catheters. In one particularly preferred embodiment, the invention relates to a docking device that can couple different types of imaging catheters to a transurethral ultrasonic imaging system.

Intraluminal, intravascular treatments, and the diagnosis of medical conditions are usually performed by using different types of imaging catheters. The imaging catheters (hereinafter referred to as catheters) are used for obtaining radial scans of a tissue, a region of interest, or an organ such as a male prostate gland. The radial scans are then used for diagnostic and other medical purposes. Typically, a catheter will be coupled to an imaging system via some type of docking apparatus. The catheter often will include an ultrasonic transducer that can be used to both generate and receive ultrasonic signals. Moreover, by directing ultrasonic waves toward a target tissue and monitoring the reflected waves that return from the target tissue and surrounding tissues, it is possible to develop image data descriptive of the target tissue. For example, ultrasonic pulses, which penetrate the periluminal tissue of an organ, will be reflected to varying degrees as they encounter tissue densities of different acoustic impedances. The reflected ultrasonic pulses are converted by the ultrasonic transducer into imaging electrical signals that are processed by the imaging system to generate an image of the organ.

Docking devices used in ultrasonic imaging systems provide mechanical and electrical interfaces for imparting axial and rotational motion to the ultrasonic transducer and facilitating the exchange of electrical signals between the catheter and the imaging system.

Although ultrasonic imaging systems are well known in the art, it remains the practice of most known imaging system manufacturers to develop catheters that are useful solely with the imaging systems that they sell. This limits the availability of competing products in the marketplace and, in turn, tends to drive up prices for the imaging systems and related catheter products that are present in the market. This is particularly true in areas such as the transurethral imaging area, where many off-the-shelf catheters could conceivably be used to affect common imaging procedures.

Because it is desirable in many applications, to use different types of catheters with the imaging system, a new docking device is desired.

### SUMMARY OF THE INVENTION

In one innovative aspect, preferred embodiments of the invention are directed to docking devices for enabling a designated imaging system to be used with a multiplicity of the catheters, independent of any relationship between the source of manufacture of the designated imaging system and the catheter. In one preferred embodiment, a docking facility (hereinafter also referred to as a “docking device”) may utilize a coupler to provide mechanical and electrical coupling between the imaging system and the catheter. The configuration of the coupler may be altered, and multiple couplers may be utilized, to facilitate mechanical and electrical communication between an imaging system and any number of catheters.

In another innovative aspect, docking facilities and couplers may be provided that are capable of monitoring various system parameters, such as total system usage, catheter end-of-life (EOL), system warranty, and catheter warranty data to ensure proper functioning of a catheter with an imaging system.

In still another innovative aspect, a docking device in accordance with various aspects of the present invention may include hardware or software for controlling the orientation of displayed images obtained upon the scanning of an organ or other tissue of interest.

Accordingly, it is an object of the present invention to provide an improved docking facility for use with various types of imaging systems, including but not limited to, transurethral ultrasonic imaging systems.

### BRIEF DESCRIPTION OF THE DRAWINGS

FIG. 1 illustrates an operational environment in which various embodiments of the present invention can be utilized.

FIG. 2 is a block diagram of a system for coupling different types of catheters to an imaging system, in accordance with one embodiment of the present invention.

FIG. 3 is a block diagram showing elements of a docking device, in accordance with one embodiment of the present invention.

FIG. 4 is a block diagram of an electronic orientation module for use with the docking device, in accordance with one embodiment of the present invention.

### DETAILED DESCRIPTION

For the sake of convenience, several terms used to describe various human anatomical structures and embodiments of the invention are defined below. It should be understood that these are provided merely to aid the understanding of the description, and that the definitions should in no way limit the scope of the invention, which is defined by the appended claims.

Anterior: Situated at the front or the front surface of an organ.

Apex of the prostate: The end of the prostate gland located farthest away from the urinary bladder.

Axial/Longitudinal: Along the centerline of the urethra, regardless of patient position.

Biopsy: The removal of a small sample or samples of tissue for examination under a microscope or other device.

Bladder: The hollow organ that stores and discharges urine from the body.

Bladder neck: The outlet area of the bladder. It is composed of circular muscle fibers (bladder sphincter), and helps control urine flow from the bladder into the urethra.

Catheter drive mechanism: A motion control system that can provide axial and/or rotational motion to an imaging catheter, or an ultrasonic transducer disposed within an imaging catheter.

Distal: Remote, farther from any point of reference (the opposite of proximal).

Genitourinary system: Pertaining to the genital and urinary systems.

Imaging catheter: A tubular mechanism, containing an ultrasonic transducer for organ-tissue imaging.

Inferior: Anatomically refers to a lower surface of an organ, or a location situated below a given reference point.

Intraluminal: Within a lumen, such as a vessel or other tubular passage within the body, an organ of the body, or an area of tissue within the body.

Introducer: A device that facilitates the insertion of a catheter into the urethra.

Periluminal: Around a lumen, such as a vessel or other tubular passage within the body, an organ of the body, or an area of tissue within the body.

Posterior: Situated at the back or the back surface of an organ.

Prostatic Urethra: The segment of the urethra, which is surrounded by prostatic tissue from the proximal end at the bladder neck to the distal end at the apex of the prostate gland.

Proximal: Closer to any point of reference.

Superior: Anatomically refers to an upper surface of an organ, or situated above a given reference point.

Transducer: A device, which transforms one form of energy to another form of energy (e.g. electrical to acoustical energy, or, conversely, acoustical to electrical energy).

Transurethral: A procedure performed through the urethra.

Transverse: Placed crosswise, situated at right angles to the long axis of an organ.

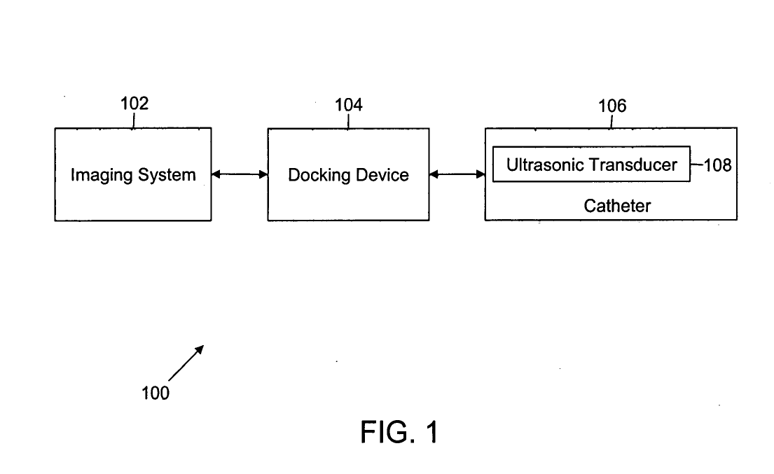

Turning to the drawings, FIG. 1 illustrates an operational environment 100 in which various embodiments of the present invention can be utilized. The operational environment 100 includes an imaging system 102, a docking device 104, and a catheter 106. The catheter 106 includes an ultrasonic transducer 108. In various embodiments of the present invention, the operational environment 100 includes a plurality of catheters.

The imaging system 102 generates and displays images of a selected anatomical region of interest, including various organs, such as a prostate gland. The imaging system 102 includes a motor or motors (not shown) for providing axial and rotational motion to the catheter 106 and/or the ultrasonic transducer 108 disposed within the catheter 106, for scanning the organ. Some examples of motors include, but are not limited to, brushed direct current motors, brushless direct current motors, stepper motors, and alternating current induction motors.

The imaging system 102 receives commands from a user for scanning a selected anatomical structure such as an organ. In one embodiment, the commands are given by pressing keys on a keypad, or with the help of any other data entry device (not shown) of the imaging system 102. In another embodiment, the commands are given through a speech recognition system that recognizes commands such as start, pause, resume, and so forth. The imaging system 102 generates electrical signals corresponding to the commands. These signals are used to control the function of the docking device 104 and the catheter 106 and for generating ultrasonic signals so that a desired image of the organ is obtained.

The docking device 104 provides an electrical and mechanical interface between the catheter 106 and the imaging system 102. The electrical interface facilitates the transfer of signals between the imaging system 102 and the catheter 106. The mechanical interface provides axial and rotational motion to the catheter 106, and/or the ultrasonic transducer 108 disposed within the catheter 106.

The catheter 106 is used to obtain radial scans of a tissue region of interest, typically a selected organ such as a male prostate gland. The scanned image of the organ is displayed on a display unit or a recording device (not shown) associated with the imaging system 102. The scanned images of the organ are used for various purposes such as to detect and differentiate between normal and abnormal tissues. For example, the catheter 106 can be used to scan the periluminal tissue of a patient during diagnosis, or the treatment of a medical condition.

The catheter 106 may include an ultrasonic transducer 108 that may be used for intraluminal scanning of an organ or any other tissue area. The ultrasonic transducer 108 is typically rotated at a uniform speed for a 360° scan of the organ. The 360° scan ensures that a complete transverse image slice of the periluminal tissue area is obtained. The ultrasonic transducer 108 scans the organ by converting electrical signals generated by the imaging system 102 and transmitted through the docking device 104 into ultrasonic pulses. The ultrasonic pulses are made to penetrate the periluminal tissue of the organ. The periluminal tissue reflects the returned echoes of the ultrasonic pulses as they encounter tissue densities of different acoustic impedances. The ultrasonic transducer 108 then converts received reflected ultrasonic pulses into imaging electrical signals. The intensity of the imaging electrical signals is proportional to the intensity and acoustic impedance of the reflected ultrasonic pulses. The imaging electrical signals are passed to the imaging system 102 through the docking device 104 and are thereafter processed to generate images of the tissue area surrounding the transducer 108.

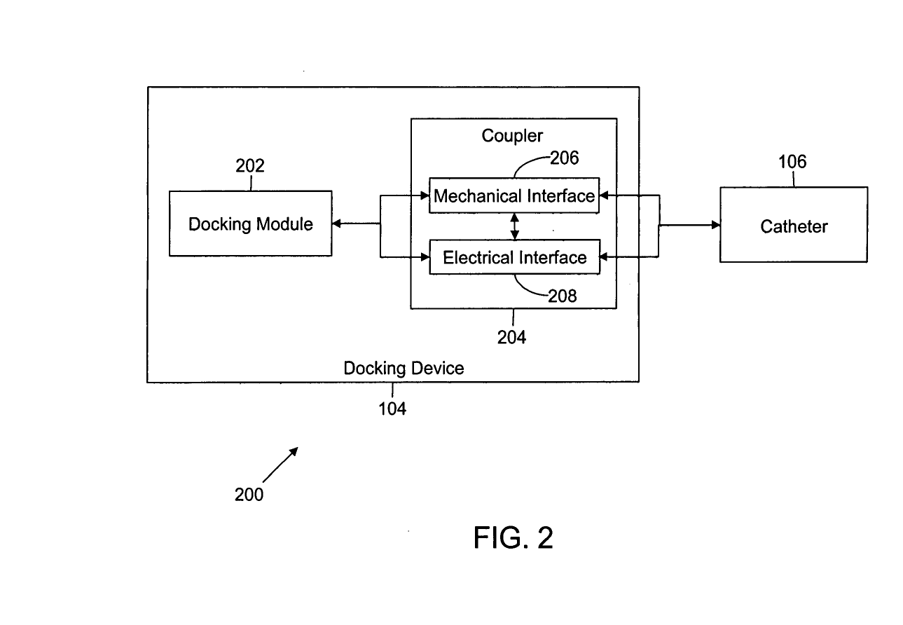

Turning now to FIG. 2, a system 200 for coupling different types of catheters to the imaging system 102 is illustrated. The system 200 includes the docking device 104 and a plurality of available catheters, hereinafter referred to as the catheter 106. The docking device 104 comprises a docking module 202 and a plurality of available couplers 204. As shown, the docking device 104 comprises a coupler 204. The coupler 204 comprises a mechanical interface 206 and an electrical interface 208.

In one embodiment, the docking module 202 is attached to the imaging system 102 and forms an integral part of the imaging system 102. In another embodiment, the docking module 202 may comprise a separate component used with the imaging system 102. It is presently preferred, that the docking module 202 comprise a separate component of the imaging system 102, and that the docking module be capable of stepping under control of the imaging system 102 in an axial direction. The docking module 202 provides a mechanical and electrical interface between the imaging system 102 and the combination of the coupler 204 and the catheter 106.

The coupler 204 enables the docking module 202 to interface with one type of catheter or another. In one embodiment, each coupler is specific to a selected catheter type. Specifically, the coupler 204 provides a mechanical and electrical coupling that enables a selected catheter 106 to be used with the docking device 104 and the imaging system 102. Similarly, the mechanical and electrical couplings engage and enable the interaction of the selected catheter 106 with the docking module 202.

The mechanical interface 206 of the coupler 204 provides a mechanical coupling between the catheter 106 and the docking module 202. The mechanical coupling facilitates the transfer of axial and rotational motion from the docking module 202 through the coupler 204 to the catheter 106 and/or to the ultrasonic transducer 108 disposed within the catheter 106. The mechanical coupling includes a catheter interface mechanism (not shown) for transferring the axial and rotational motion to the catheter 106. The catheter interface mechanism is a motion control system that provides axial and/or rotational motion to a catheter. In one embodiment, the catheter interface mechanism provides linear and translational motion to the catheter 106. In one embodiment, the mechanical coupling of the mechanical interface 206 is made flexible to allow the easy movement of the catheter 106. The electrical interface 208 of the coupler 204 provides an electrical coupling between the catheter 106 and the docking module 202. The electrical coupling preferably facilitates the bi-directional passage of electrical signals between the docking module 202 and the catheter 106 through the coupler 204. The electrical signals are exchanged between the docking module 202 and the catheter 106 for scanning the periluminal tissue of the organ and for collecting the catheter usage data from the catheter 106. The interconnect between the mechanical interface 206 and the electrical interface 208 facilitates the transfer of motion and bi-directional electrical signals between the docking module 202 and the catheter 106.

In one embodiment, the mechanical interface 206 and the electrical interface 208 of the coupler 204 are specific to a selected catheter 106 and provide electrical and mechanical couplings specific to the requirements of the catheter 106.

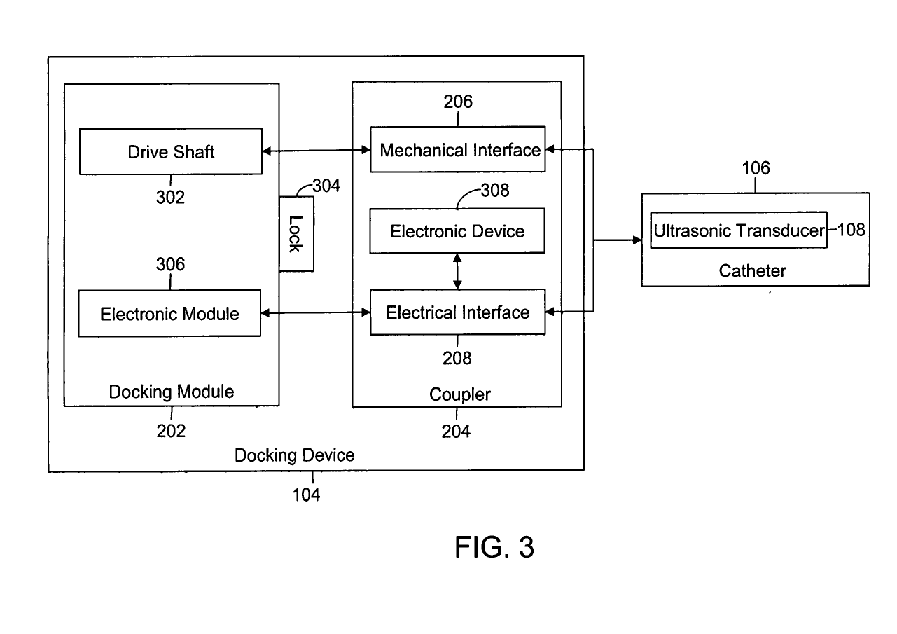

Turning to FIG. 3, in a preferred form of the present invention, additional elements of the docking device 104 are illustrated. As shown, the docking device 104 includes the docking module 202 and the coupler 204. The docking module 202 includes a drive shaft 302, a lock 304, and an electronic module 306. The coupler 204 includes the mechanical interface 206, an electronic device 308, and the electrical interface 208.

The drive shaft 302 transfers rotational motion of the motor in the imaging system 102 to the mechanical interface 206. The mechanical interface 206 transfers the rotational motion to the catheter 106 and/or the ultrasonic transducer 108 disposed within the catheter 106. The mechanical coupling of the mechanical interface 206 converts the rotational motion corresponding to the mechanical requirement of the catheter 106. The mechanical coupling comprises a plurality of accessory mechanical coupling devices for this conversion. The converted rotational motion is then transferred to the catheter 106.

The lock 304 locks the docking module 202 with the coupler 204, i.e., the lock 304 retains the coupler 204 tightly to the docking module 202. Examples of locks include bolts, locking pins, screws, etc.

In one presently preferred embodiment, the electronic module 308 may store system information such as system serial number, date of first use, overall system runtime, system serial number, warranty and historical information and other such system dependent or catheter independent data. Electronic module 308 also may store catheter usage data, catheter type and serial number data, catheter “end of life” (EOL) data, and other catheter dependent data.

In one embodiment of the present invention, the electronic device 308 generates a warning signal of the catheter 106 EOL when the usage the catheter 106 approaches its EOL. The electronic device 308 further locks the catheter 106 on expiry of the catheter 106 end-of-life, thereby preventing the further usage of the catheter 106. In another embodiment of the present invention, the electronic module 306 sends to and retrieves from the electronic device 308 data enabling the electronic module 306 to make the catheter EOL control decisions.

In one embodiment, the docking module 202 also includes a circuitry for controlling the orientation of an image scanned by the catheter 106. The superior side of an organ is the upper surface of the organ. The orientation of the image is controlled so that a superior (upper) side of the organ is displayed in the uppermost position on the display unit of the imaging system 102. The importance of displaying a properly oriented image on the display is to aid a user of the imaging system 102 in understanding the image being displayed and to assist a user of the imaging system 102 in carrying out a medical procedure. The circuitry controls the orientation of the image in this embodiment by delaying an index pulse. The index pulse is a signal generated by the electronic module 306 to notify the imaging system 102 about the completion of a 360° rotation by the catheter 106, and the beginning of the next rotation i.e., the index pulse indicates that the ultrasonic transducer 108, which in this embodiment, may be continuously rotating, has reached the point of start of the next scan. The point of start of the scan is marked for reference. The imaging system 102 begins each scan on receiving the index pulse. The delay in the index pulse leads to a delay in starting the scan capturing of 360° image data relative to the physical ‘top’ of the rotating catheter 106 by the imaging system 102, thereby rotating the image about its center. The image is rotated until the superior (upper) side of the organ is displayed upright on the display unit of the imaging system 102.

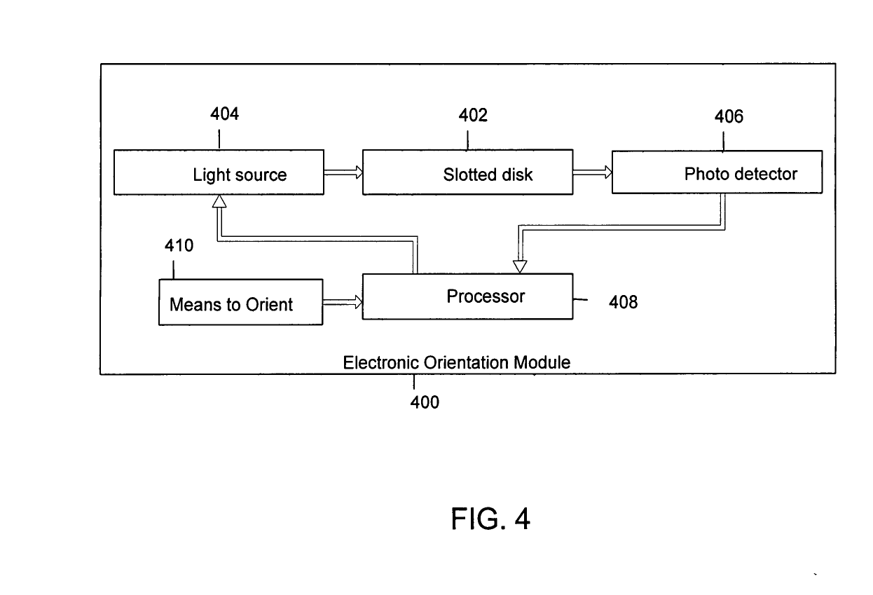

Turning to FIG. 4, in a preferred form, an electronic orientation module 400 is used with the docking device 104. The electronic orientation module 400 controls the orientation of the display of an image by the imaging system 102 of an organ scanned by the catheter 106. This enables the upright display of the superior portion of the organ on the display unit of the imaging system 102. In this embodiment, the electronic orientation module 400 comprises a slotted disk 402, a light source 404, a photo-detector 406, a micro-processor 408 for controlling the orientation process, and a means, typically including a potentiometer and/or software or programming instructions, for setting the orientation 410 of the image scanned.

The slotted disk 402 is mounted on the drive shaft 302 of the docking module 202. The slotted disk 402 is an opaque circular disk with a slot (or in alternate embodiments a small hole or other transparent shape) on its periphery. The light source 404 is located on one side of the slotted disk 402 and the photo-detector (receptor) 406 is located on the opposing side of the slotted disk 402.

A light beam from the light source 404 is made to strike the surface of the opaque slotted disk 402. The photo-detector 406 is arranged to detect the light beam from the light source 404. The surface of the slotted disk 402 prevents the light beam from reaching the photo-detector 406. However, the light reaches the photo-detector 406 when the slot on the slotted disk 402 comes in between the light source 404 and the photo-detector 406 after every 360° rotation of the drive shaft 302. The photo-detector 406 detects the light beam which is received as a signal by the imaging system 102. This signal notifies the imaging system 102 about the completion of a 360° rotation of the drive shaft 302. In one embodiment, the photo-detector 406 outputs a signal to a processor 408, a part of the electronic orientation module, and the means to orient 410 notifies the processor 408 how much delay if any to delay its output. At the designated time, the electronic orientation module processes the signal and forwards it to the imaging system 102. The processor 408 has programming instructions for controlling the orientation of the image of the organ. In one embodiment, the processor 408 is a part of the imaging system 102. The processor 408 uses the signal along with the programming instructions and the means to orient 410 for enabling the upright display of the superior portion of the imaged organ on the display unit of the imaging system 102. An alternative embodiment to utilizing the slotted disk 402, light source 404 and the photo-detector 406 would be to utilize an electronic encoder often found as an integral part of or an add-on to a motor.

The means to orient 410, in one embodiment is a potentiometer or other adjustable component which varies a signal to the processor 408. This adjustment may be accomplished by manual or automatically controlled mechanical means, by electronically controlled means or other means commonly known to one skilled in the art. The processor 408 interprets this signal received from the means to orient 410 and interprets using it's own internal programming to create the desired (if any) delay prior to forwarding the index signal to the imaging system 102.

While the invention is susceptible to various modifications and alternative forms, specific examples thereof have been shown by way of example in the drawings and are herein described in detail. It should be understood, however, that the invention is not to be limited to the particular forms or systems disclosed, but to the contrary, the invention is to cover all modifications, equivalents, and alternatives falling within the spirit and scope of the appended claims.

What is claimed is:

1. A facility for coupling different types of catheters to an imaging system, the facility comprising:
a docking module for providing a mechanical and electrical interface to a selected catheter; and
a plurality of accessory couplers for enabling a plurality of additional catheters having different mechanical and electrical coupling structures to engage and interact with the docking module.

2. The facility of claim 1 wherein the docking module comprises an electronic module for storing catheter usage data.

3. The facility of claim 1 wherein the docking module comprises an electronic module for storing system usage data.

4. The facility of claim 2 wherein the docking module further comprises a drive shaft for transferring rotational motion of a motor to a catheter interface mechanism and a coupler that engages the catheter interface mechanism.

5. The facility of claim 2 wherein the docking module further comprises a lock for locking the docking module with a coupler.

6. The facility of claim 1 wherein a coupler comprises an electronic device for storing catheter usage data.

7. The facility of claim 6 wherein the coupler allows exchange of electronic signals between the docking module and the catheter.

8. The facility of claim 6 wherein the electronic device generates a catheter end of life warning signal when the catheter usage approaches the catheter end of life.

9. The facility of claim 7 wherein the electronic device locks the catheter on expiry of the catheter end of life, preventing further usage of the catheter.

10. A coupler for use in an ultrasonic imaging system, the coupler comprising: a mechanical interface for providing a mechanical coupling between a docking module and a catheter, and
a electrical interface for providing an electrical coupling between the docking module and the catheter.

11. A docking device for use with an imaging system, the system comprising:
a docking module for providing an electrical and mechanical interface to a catheter; and
a plurality of couplers for enabling a plurality of catheters having different mechanical and electrical coupling structures to engage and interact with the docking module wherein the docking module comprises:
means for controlling rotation of a catheter engaged with the docking module through a selected coupler;
means for communicating electrical signals transmitted to and received from the catheter to an imaging module; and
means for monitoring usage data selected from the group consisting of either system usage data or catheter usage data.

12. An electronic orientation module for use with a catheter docking section of an imaging system, the mechanism comprising:
a slotted disk mounted to a drive shaft of a catheter rotational drive system;
a light source located to one side of the slotted disk;
a photo-detector located on an opposing side of the slotted disk, and arranged to detect light from the light source as a slot formed in the slotted disk passes between the light source and the photo-detector; and
a processor located in an associated imaging system having programming instructions for interpreting signals received from the photo-detector and using the signals received from the photo-detector to enable upright display of a superior portion of an imaged organ on a display unit of the imaging system.
